(19) United States
(12) Patent Application Publication
(10) Pub. No.: US 2025/0073058 A1
(43) Pub. Date: Mar. 6, 2025

(54) ANTI-SNORING APPLIANCE THAT REPOSITIONS TONGUE AND MANDIBLE

(71) Applicant: Thomas Patrick Roemer, Bettendorf, IA (US)

(72) Inventor: Thomas Patrick Roemer, Bettendorf, IA (US)

(21) Appl. No.: 18/823,166

(22) Filed: Sep. 3, 2024

Related U.S. Application Data
(60) Provisional application No. 63/580,116, filed on Sep. 1, 2023.

(51) Int. Cl. A61F 5/56

(52) U.S. Cl. CPC A61F 5/566

(57) ABSTRACT

An anti-snoring appliance that repositions the tongue and mandible, reducing and/or eliminating snoring and sleep apnea, has two primary points of contact with the tongue. The appliance depresses, lifts, and secures the tongue and repositions the mandible at the same time. This movement of both the tongue and the mandible improves airflow while the user sleeps.

### CROSS-REFERENCE TO RELATED APPLICATION

This application claims the benefit of priority of U.S. provisional application No. 63/580,116, filed Sep. 1, 2023, the contents of which are herein incorporated by reference.

### BACKGROUND OF THE INVENTION

The present invention relates to dentistry appliances, mouthpiece-type retainers, and mouthguards, and more particularly, to anti-snoring appliances.

Many factors can contribute to what causes airflow obstructions during sleep, and the intensity of the snoring. Regardless of the factors, the permeating sounds can be a disturbance to those awake and a health concern to the individual who snores. Some of the contributing factors can be related to the size and shape of the muscles and tissues in the head and neck area; including but not limited to the anatomy of a person's mouth, throat, and sinuses.

There are many anti-snoring appliances currently on the market. There are two main types of anti-snoring mouth appliances: Mandibular advancement devices (MADs) and tongue-stabilizing devices (TSDs). Mandibular advancement devices are the most common devices for treating airflow obstructions. MADs work by pulling your lower jaw and tongue to a more forward position creating more space for airflow. TSDs pull your tongue forward using different techniques. Like the MADs, the TSDs open your airway by moving the base of the tongue forward. The more effective appliances are usually custom-made to fit the user's mouth. Very few appliances effectively isolate specific movements of the tongue and its surrounding soft tissues. Most of the MAD and TSD appliances currently on the market are uncomfortable and difficult to keep in your mouth throughout the night, thereby rendering them less effective. CPAP machines may be an alternative treatment for snoring and sleep apnea, but they are not without their downfalls. These machines have a positive pressure airflow which can cause ulcers in the mouth and can be noisy and uncomfortable to wear.

When you combine reduced airflow and relaxed soft tissues in and around the nasopharynx, you get snoring and sleep apnea. The oral appliance reduces movement between soft tissues around the nasopharynx; typically the soft palate and the tongue.

As can be seen, there is a need for anti-snoring appliances that can negate the effects of snoring by implementing lower jaw and tongue displacement simultaneously.

### SUMMARY OF THE INVENTION

In one aspect of the present invention, an anti-snoring dental appliance comprises a lower guard including an occlusal surface with a buccal-labial flange extending therefrom and with a lingual flange extending therefrom, the occlusal surface, buccal-labial flange, and lingual flange forming a substantially U-shaped channel therebetween configured to accommodate a user's lower teeth, the U-shaped channel having a first end and a second end; a posterior strap joining a first portion of the lingual flange proximal to the first end with a second portion of the lingual flange proximal to the second end; and a flap extending from a central portion of the lingual flange.

The anti-snoring appliance of the present subject matter repositions the tongue and the mandible so that they are in a more favorable position slightly further away from the soft palate and the back of the throat. It locks and holds the tongue in place better than any other product on the market. The appliance, which the user wears in the mouth, keeps the airway open while the user sleeps. Repositioning of the tongue and mandible can create additional airflow, lessening or even eliminating snoring and sleep apnea.

Since the invention has the ability to reproduce a particular movement without conscious thought, the tongue may become trained to stay in the “locked” position while sleeping. This newly acquired muscle memory may mean the user will no longer need to wear the invention on a daily basis; since they will experience less or no snoring or sleep apnea.

These and other features, aspects and advantages of the present invention will become better understood with reference to the following drawings, description, and claims.

### BRIEF DESCRIPTION OF THE DRAWINGS

FIG. 1 is a perspective view of an anti-snoring device according to an embodiment of the present invention, shown in use;

FIG. 2 is a sectional view thereof, taken along line 2-2 in FIG. 1;

FIG. 3 is another sectional view thereof, taken along line 3-3 in FIG. 2;

FIG. 4 is a cut away, sectional view thereof;

FIG. 5 is a perspective view of an anti-snoring device according to another embodiment of the present invention;

FIG. 6 is a perspective view of an anti-snoring device according to another embodiment of the present invention, shown in use:

FIG. 7 is a sectional view thereof, taken along line 7-7 in FIG. 6; and

FIG. 8 is another sectional view taken thereof along line 8-8 in FIG. 7.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, one embodiment of the present invention is an anti-snoring appliance that reduces or eliminates snoring by properly repositioning the tongue and lower mandible. The anti-snoring appliance comprises two primary points of contact with the tongue, which depress and lift the tongue at the same time.

The anti-snoring appliance depresses the tongue in one area and lifts and secures the tongue in another area. This dual action moves the tongue away from other soft tissues in the throat and reduces movement between soft tissues around the nasopharynx, typically the soft palate and tongue. The anti-snoring appliance can move the lower jaw further away from the back of the throat to a more forward position with the addition of the mandibular advancement layer, thus improving airflow, i.e., allowing air to flow more freely and uninterrupted. The anti-snoring appliance is used to reduce or eliminate snoring and/or sleep apnea.

This appliance has a lower guard with two primary components-a strap or bar and a ramp or flap-which can work together to depress and lift different regions of the tongue at the same time. This dual action moves the tongue away from the back of the throat to a position that improves airflow and reduces snoring.

The strap/bar depresses the middle or posterior portion of the tongue. The strap/bar may be altered in size, shape, and/or position to provide more comfort for the consumer.

The anterior ramp/flap aids in repositioning the tongue. This ramp/flap may turn the anterior portion of the tongue in the upward/posterior position, thus securing the tongue in place against the strap. Alternatively, the flap may lie about level with the mandibular portion of the device surrounding the user's lower teeth or may be turned downward within the mouth of the cavity between the base of the tongue and the musculature at the bottom of the mouth. The latter configuration raises the middle of the tongue towards the roof of the mouth, while allowing the tip of the tongue to lie substantially flat within the mouth. Both components help move the tongue to a position that enables better airflow.

The appliance also may have an additional layer of material that spaces the lower guard from the upper guard/teeth, thereby aiding in the movement of the lower mandible anteriorly.

For optimal fitting, the appliance may be professionally fabricated at a certified lab by a skilled dental laboratory technician and custom fitted by the Dentist who is treating the user. The appliance can also be a prefabricated kit, supplied at-home, including a component with a flap and strap that can be hot-water boiled or heated prior to the user placing it in his/her mouth and biting down to record the upper and lower dentition. The component may then be worn immediately (“boil and bite” technique). Alternatively, the consumer may make impressions of their teeth and surrounding areas using a kit supplied by a company and send the impressions back to the company for fabrication of the appliance. Once fabricated, the appliance will be shipped to the consumer for use.

The material used to fabricate the appliance can be hard or soft in nature. The material is non-toxic and compatible for use in a human subject's mouth and sufficiently rigid to retain its shape under pressure exerted by a person's normal wear and tear. Hard plastics used in orthodontic appliances are known in the art and can be used in the present appliance. Thermoplastics may be used, i.e., a polymer material which may be softened by heat and hardened by cooling in a reversible physical process. Any portion of the appliance which contacts the tongue or soft tissues is preferably rounded, shaped so that there are no corners or sharp edges for comfort to the user and to avoid injury to the soft tissues of the oral cavity.

An impression or duplicate of the upper and lower dentition (i.e., teeth) may or may not include partial anatomy of the lower jawbone and tongue. The duplicate may be recorded either physically by an impression or digitally with a scanner. The area in which the tongue will be depressed or lifted may be modified. The lower guard may have at least one strap.

To use the appliance described herein, the person may wear the appliance whenever he or she goes to sleep, thereby eliminating or reducing snoring to an acceptable, healthy, stress-free level.

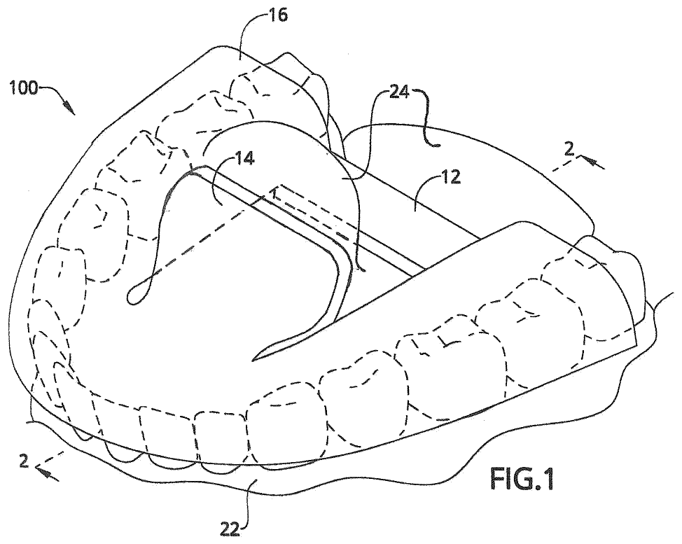

Referring to the Figures, FIG. 1 illustrates an anti-snoring device 100 comprising a lower guard 16 having a channel that fits around/covers the dentition in the user's lower mandible 22. The lower guard 16 includes a translingual strap 12 across a posterior portion of the lower guard 16 which extends from the lingual surface of the right molars to the lingual surface of the left molars and a flap 14 extending from the anterior center of the guard 16 in a posterior direction which, in combination with the channel, retain the lower guard 16 in place in the user's mouth. The strap 12 depresses the posterior portion of the tongue 24, primarily keeping the tongue 24 away from the soft palate and the back of the throat. The flap 14 holds the user's tongue 24 in a raised position such that the user's airway is unobstructed. As shown in FIG. 1, the flap 14 raises the anterior portion of the tongue 24 away from the floor of the mouth to a position where it is secured by the strap 12.

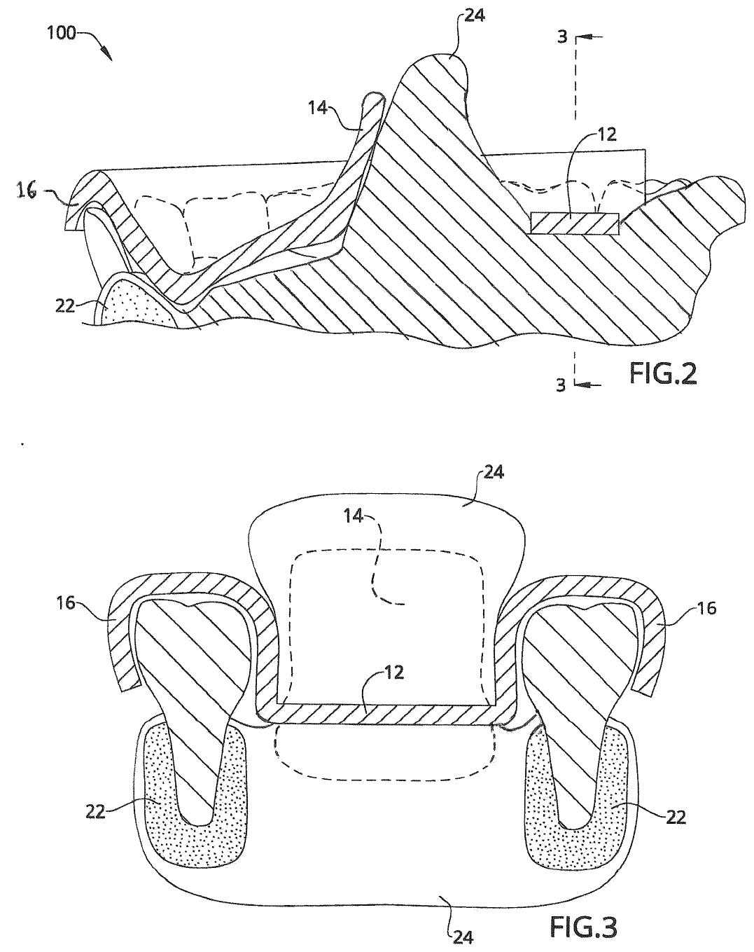

FIGS. 2 and 3 illustrate the flap 14 in an inclining position, guiding the tongue 24 upward.

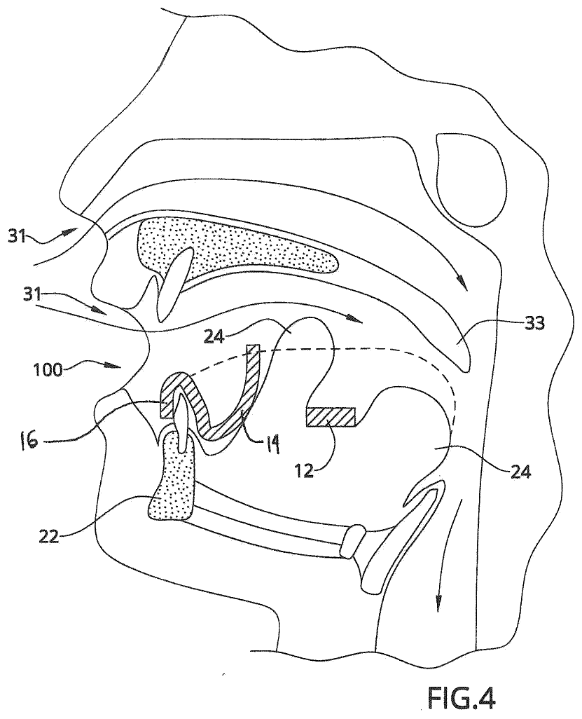

As FIG. 4 demonstrates, the strap 12 retains the tongue 24 in a position spaced from the soft palate 33 that allows air 31 to pass through the user's airway unobstructed.

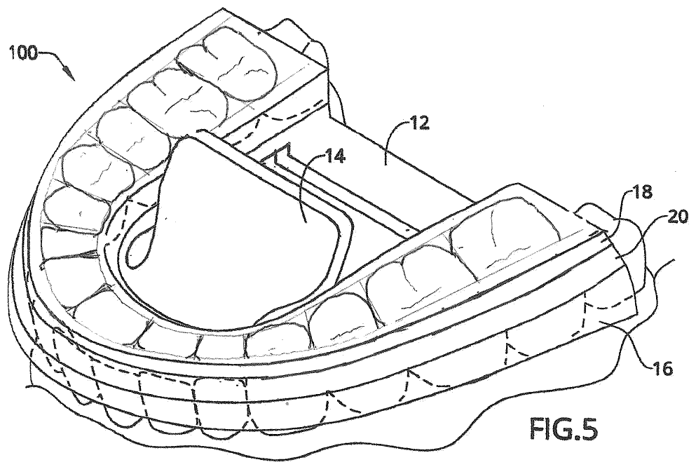

FIG. 5 illustrates an embodiment of the device 100 also having an upper guard 18 featuring a mandible advancement layer 20 which, when worn in conjunction with the lower guard 16, urges the user's lower mandible 22 forward into a more anterior position, opening the airway even further. The mandibular advancement layer aids in positioning the two guards in a static position while moving the lower mandible to a more anterior position.

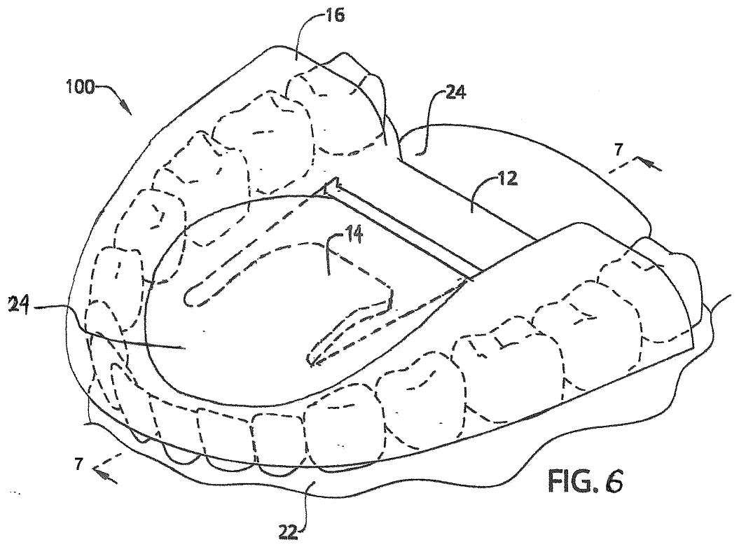

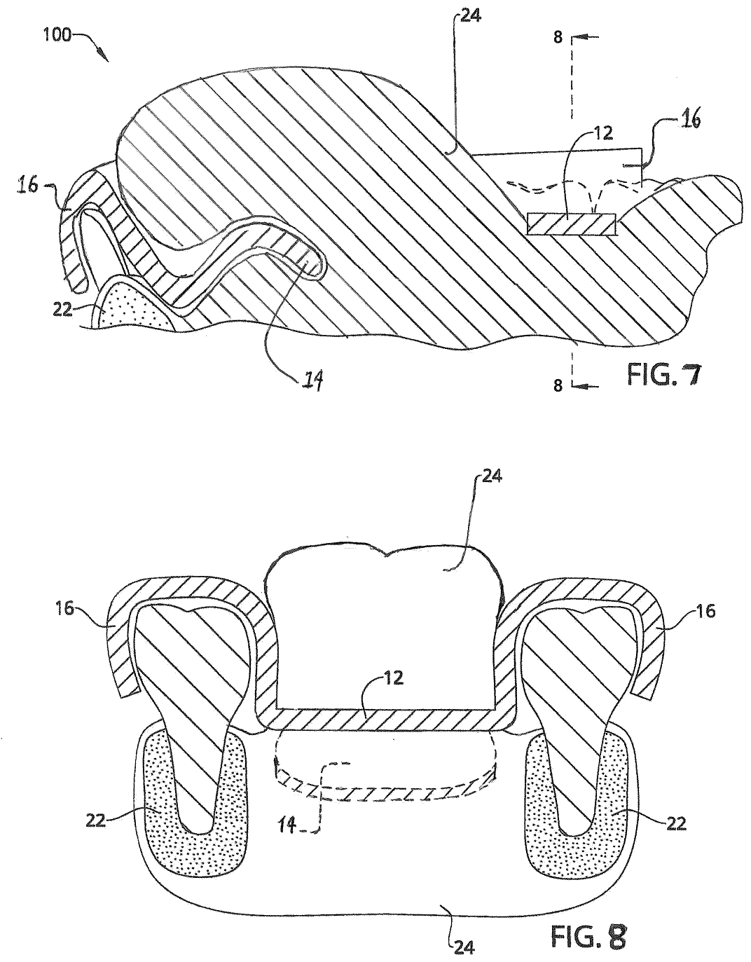

The flap 14 is not limited to the position shown in FIGS. 1 through 5. FIGS. 6 through 8 illustrate an embodiment of the anti-snoring device 100 having a flap 14 that guides the tip of the user's tongue 24 in a downward direction to a lowered position. As in FIGS. 1 through 3, a lower guard 16 has a channel that fits around/covers the dentition in the user's lower mandible 22. A translingual strap 12 across a posterior portion of the lower guard 16 extends from the lingual surface of the right molars to the lingual surface of the left molars and depresses the posterior portion of the tongue 24. The flap 14 extends from the anterior center of the guard 16 in a posterior direction.

It should be understood, of course, that the foregoing relates to exemplary embodiments of the invention and that modifications may be made without departing from the spirit and scope of the invention as set forth in the following claims.

What is claimed is:

1. An anti-snoring dental appliance, comprising a lower guard including:
(a) an occlusal surface with a buccal-labial flange extending therefrom and with a lingual flange extending therefrom, the occlusal surface, buccal-labial flange, and lingual flange forming a substantially U-shaped channel therebetween configured to accommodate a user's lower teeth, the U-shaped channel having a first end and a second end;
(b) a posterior strap joining a first portion of the lingual flange proximal to the first end with a second portion of the lingual flange proximal to the second end; and
(c) a flap extending from a central portion of the lingual flange.

2. The anti-snoring dental appliance of claim 1, wherein the flap curves to form a ramp against which an anterior portion of the user's tongue rests when the lower guard is placed in the user's mouth.

3. The anti-snoring dental appliance of claim 1, wherein the flap arches to rest under the tongue when the lower guard is placed in the user's mouth.

4. The anti-snoring dental appliance of claim 1, wherein the lower guard has a mandibular advancement layer forming a flat surface opposite the occlusal surface.

5. The anti-snoring dental appliance of claim 1, further comprising an upper guard, configured to accommodate the user's upper dentition, having a mandibular advancement layer operative to urge the user's lower mandible anteriorly, and wherein the upper guard aligns with the lower guard and is configured to prevent movement of the upper guard relative to the lower guard.
